(19) United States
(12) Patent Application Publication
(10) Pub. No.: US 2024/0392682 A1
(43) Pub. Date: Nov. 28, 2024

(54) AUTOMATED METHODS FOR ESTIMATING DIFFERENTIAL LAG TIMES WHILE DRILLING

(71) Applicant: Schlumberger Technology Corporation, Sugar Land, TX (US)

(72) Inventors: Reda Karoum, Houston, TX (US); Steven Philip Young, Cypress, TX (US); Mahdi Ammar, Clamart (FR); Karim Bondabou, Montpellier (FR)

(21) Appl. No.: 18/672,601

(22) Filed: May 23, 2024

Related U.S. Application Data
(60) Provisional application No. 63/503,739, filed on May 23, 2023.

(51) Int. Cl. E21B 47/11; E21B 21/06; E21B 21/08; E21B 45/00

(52) U.S. Cl. CPC E21B 47/11; E21B 21/065; E21B 21/08; E21B 45/00

(57) ABSTRACT

A method for estimating a differential lag time while drilling includes introducing a multiphase tracer into drilling fluid circulating in a wellbore while drilling. The multiphase tracer includes a solid tracer and a fluid tracer in which the fluid tracer includes at least one of a liquid tracer or a gaseous tracer. A first arrival time of the solid tracer and a second arrival time of the fluid tracer are measured at a surface location and evaluated to estimate the differential lag time. The differential lag time includes at least one of a difference between a cuttings lag time and a gaseous lag time or a difference between the cuttings lag time and a liquid lag time.

### CROSS REFERENCE TO RELATED APPLICATIONS

None

### BACKGROUND

Drilling fluid (mud) is pumped downhole while drilling a subterranean wellbore. The fluid emerges from the drill string at the drill bit and creates an upward flow through the wellbore annulus which carries drill cuttings to the surface. The fluid and cuttings are commonly examined at the surface to evaluate the formation layers though which the wellbore is drilled. The depth at which the cuttings were generated may be determined from a depth log generated while drilling and the lag time of the cuttings (the time it takes the cuttings to reach the surface).

A theoretical lag time may be calculated from the well architecture (e.g., including the wellbore and drill string diameters) and the fluid flow rate. However, in practice the theoretical lag time does not provide a consistently accurate measure of the cuttings lag time. As such, drilling operators commonly make occasional lag time measurements while drilling a well. For example, a drilling operator may introduce particulate (e.g., rice grains) into the drilling fluid and estimate the lag time based on the arrival time of the particulate at the surface. Alternatively, a drilling operator may introduce acetylene gas or calcium carbide into the well and estimate the lag time based on the arrival time of the gas at the surface.

While the above described methods for determining the lag time may at times be commercially serviceable, there is room for further improvement. For example, identifying the introduced particulate amidst the drill cuttings in the arriving drilling fluid can be difficult such that the estimated cuttings lag time may be subject to error. Moreover, the drilling process may be temporarily stopped from time to time. When stopped, the cuttings begin to settle in the annulus at a settling rate (or rates) that depend on the density, size, and shape of the cuttings particles as well as the viscosity of the mud. Moreover, formation gases (and other gases) may rise towards to the surface. As a result, the cuttings lag time and the gas or liquid lag time can diverge from one another, sometimes significantly depending on the well depth and stoppage time(s). There is a need in the industry for improved lag time measurement methods.

### BRIEF DESCRIPTION OF THE DRAWINGS

For a more complete understanding of the disclosed subject matter, and advantages thereof, reference is now made to the following descriptions taken in conjunction with the accompanying drawings, in which:

FIG. 1 depicts an example drilling rig including a system for estimating a differential lag time while drilling.

FIG. 2 depicts a flow chart of one example method for estimating a differential lag time while drilling.

FIG. 3 depicts one example multiphase tracer.

FIG. 4 depicts a flow chart of another example method for estimating a differential lag time while drilling.

FIG. 5 depicts an example method for determining an arrival time of solid tracer particles at the surface.

FIG. 6 depicts a plot of solid tracer counts on the lefthand vertical axis and tracer gas concentration on the righthand vertical axis versus time on the horizontal axis.

### DETAILED DESCRIPTION

Embodiments of this disclosure include methods and systems for estimating a differential lag time while drilling. In one example embodiment, a disclosed method includes introducing a multiphase tracer into drilling fluid circulating in a wellbore while drilling. The multiphase tracer includes a solid tracer and a fluid tracer in which the fluid tracer includes at least one of a liquid tracer or a gaseous tracer. A first arrival time of the solid tracer and a second arrival time of the fluid tracer are measured at a surface location and evaluated to estimate the differential lag time. The differential lag time includes at least one of a difference between a cuttings lag time and a gaseous lag time or a difference between the cuttings lag time and a liquid lag time.

Example ones of the disclosed embodiments may advantageously enable the bit to surface lag time for solids, liquids, and gas (as well as differential lag times therebetween) to be estimated, which may in turn enable transport issues to be identified for a given set of operational conditions (e.g., well architecture, drilling parameters, and geological formation layers). As described in more detail below, the disclosed embodiments make use of a multiphase tracer that includes both a solid tracer and a fluid (liquid and/or gas) tracer.

Depending on the drilling fluid, drill string, borehole geometry and other factors, newly produced cuttings and the drilling fluid may travel up the annulus at different rates. In certain circumstances, this can result in a net accumulation of cuttings in the annulus, which in turn can further cause hole cleaning problems, higher fluid column weight and a corresponding higher equivalent static density (ESD), and ultimately in wellbore consolidation issues, such as excessive wellbore size, cavings, stuck pipe or tools, and other drilling issues. Moreover, gases, such as formation gases, may also travel up the annulus at a different rate than both the solid cuttings and the liquid phase drilling fluid (e.g., when the gas concentration exceeds its solubility in the drilling fluid).

Lag time from the surface to the bit is commonly predicted from the volume of the drill string and the flow rate (or pressure) in the drill string. Lag time from the bit back up to the surface can also be estimated theoretically (e.g., from the volume of the annulus and the flow rate), however, such estimates are fraught with problems as described above. As also described above, drilling operators commonly add foreign material to the drilling fluid to estimate the lag time. For example, solid particulate can be added to the drilling fluid at the surface. A total lag time can be estimated from the arrival of the particulate back at the surface (after traversing the well). The lag time from the bit to the surface can be inferred by subtracting a theoretical lag time from the surface to the bit from the total lag time. While these methods can be serviceable, there is room for further improvement.

For example, there is a need to evaluate solid (e.g., cuttings), liquid (e.g., drilling and formation fluid), and gaseous (e.g., formation gases) lag times as well as differential lag times therebetween. While the liquid and gas lag times are commonly similar or even nearly identical, they often deviate significantly from the cuttings (or solid) lag time. In certain operations, evaluating a difference between solid and liquid lag times may enable an operator to identify conditions that can lead to hole cleaning problems. For example, a large positive difference between solid and liquid lag times may indicate that the cuttings are travelling up the annulus at a significant lower rate than the drilling fluid, which may in turn lead to hole cleaning (and other corresponding) problems.

FIG. 1 depicts an example drilling rig 20 including a system for estimating differential lag times while drilling (e.g., between cuttings and gas or between cuttings and the drilling fluid). The drilling rig 20 may be positioned over a subterranean formation (not shown). The rig may include, for example, a derrick and a hoisting apparatus (also not shown) for raising and lowering a drill string 30, which, as shown, extends into wellbore 40 and includes, for example, a drill bit 32 and one or more downhole measurement tools 38 (e.g., a logging while drilling tool or a measurement while drilling tool). Suitable drilling systems, for example, including drilling, steering, logging, and other downhole tools are well known in the art.

Drilling rig 20 further includes a surface system 50 for controlling the flow of drilling fluid used on the rig (e.g., used in drilling the wellbore 40). In the example rig depicted, drilling fluid 35 is pumped downhole (as depicted at 92) via a conventional mud pump 57. The drilling fluid 35 may be pumped, for example, through a standpipe 58 and mud hose 59 in route to the drill string 30. The drilling fluid 35 typically emerges from the drill string 30 at or near the drill bit 32 (e.g., via drill bit jets) and creates an upward flow 94 of mud through the wellbore annulus 42 (the annular space between the drill string and the wellbore wall). The drilling fluid then flows through a return conduit 52 and solids control equipment 55 (such as a shale shaker) to a mud pit 56 (or mud pit system including multiple mud pits). It will be appreciated that the terms drilling fluid and mud are used synonymously herein.

The circulating drilling fluid 35 is intended to perform many functions while drilling, one of which is to carrying drill cuttings 45 to the surface (in upward flow 94). The cuttings are commonly removed from the returning mud via a shale shaker 55 (or other similar solids control equipment) in the return conduit (e.g., immediately upstream of the mud pits 56). The drilling fluid 35 is generally reused and recirculated downhole. Formation gases that are released during drilling (along with optional tracer gases as described in more detail below) may also migrate to the surface in the circulating drilling fluid. These gasses are commonly removed from the fluid, for example, via a degasser or gas trap 54 located in or near a header tank 53 that is immediately upstream of the shale shaker 55 in the example depiction. The cuttings 45 and gas are commonly examined at the surface to evaluate the formation layers though which the wellbore is drilled. The depth at which the cuttings and/or gases were generated may be determined from the depth log generated while drilling and the lag time (the time it takes cuttings to reach the surface).

The disclosed embodiments include methods and systems for estimating lag times for drill cuttings, drilling fluid, and/or formation gases that are transported to the surface. For example, the disclosed embodiments may enable a differential lag time to be estimated. By differential it is meant the difference between first and second lag times corresponding to first and second distinct phases in the drilling fluid. For example, a differential lag time may be indicative of the difference between a first lag time of drill cuttings and a second lag time of formation gas. In another example, a differential lag time may be indicative of a difference between a first lag time of drill cuttings and second lag time of a liquid component in the drilling fluid (e.g., the water or oil in water-based or oil-based drilling fluid). As described in more detail below, disclosed methods employ a multiphase tracer including a solid tracer and a fluid (gaseous and/or liquid) tracer. Note also that FIG. 1 further depicts a plurality of multiphase tracer particles 200 being introduced (e.g., injected) into the drilling fluid at an injection port 51 located at the surface (e.g., between the pump and the drill string).

The rig 20 may include a system 70 configured to automatically or semi-automatically estimate the aforementioned lag times and/or differential lag times based on various cuttings, fluid, and/or gas measurements. The system 70 may be deployed at the rig site (e.g., in an onsite laboratory 60) or offsite. The disclosed embodiments are not limited in this regard. In example embodiments, the system 70 may include computer hardware and software configured to automatically or semi-automatically evaluate digital images (photographs) of the cuttings as well as gas composition measurements. To perform these functions, the hardware may include one or more processors (e.g., microprocessors) which may be connected to one or more data storage devices (e.g., hard drives or solid-state memory). As is known to those of ordinary skill, the processors may be further connected to a network, e.g., to receive the images from a networked camera system (not shown) or another compute system. It will, of course, be understood that the disclosed embodiments are not limited the use of or the configuration of any particular computer hardware and/or software.

While FIG. 1 depicts a land rig 20, it will be appreciated that the disclosed embodiments are equally well suited for land rigs or offshore rigs. As is known to those of ordinary skill, offshore rigs commonly include a platform deployed atop a riser that extends from the sea floor to the surface. The drill string extends downward from the platform, through the riser, and into the wellbore through a blowout preventer (BOP) located on the sea floor. The disclosed embodiments are not limited in these regards.

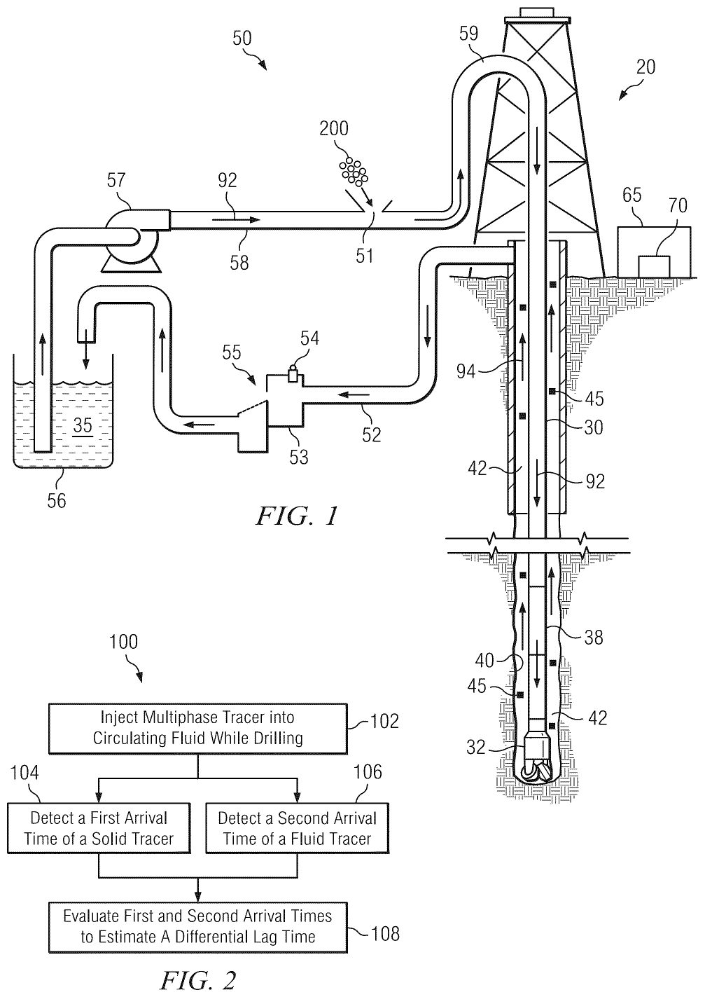

FIG. 2 depicts a flow chart of one example method 100 for estimating a lag time or differential lag time while drilling. The method includes introducing (e.g., injecting) a multiphase tracer into the circulating drilling fluid at 102 (e.g., at or just before the fluid is pumped downhole). As described in more detail below the multiphase tracer may include substantially any suitable tracer including a solid tracer and a fluid tracer in which the fluid tracer may include a liquid tracer and/or gaseous tracer. For example, the multiphase tracer may include a solid tracer (including solid particulate such as glass or ceramic beads) and a fluid tracer that decomposes or evaporates into a gas upon contact with the drilling fluid or upon the mechanical or crushing action of the drill bit. Example fluid tracers (including liquid and gas tracers) are described in more detail below. In another embodiment the multiphase tracer may include a solid tracer and a liquid tracer that remains liquid in the drilling fluid or dissolves in the drilling fluid. Method 100 further includes measuring a first arrival time of the solid tracer at the surface at 104 and measuring a second arrival time of the fluid tracer (e.g., the liquid or gas tracer) at the surface at 106. The first and second arrival times are then evaluated at 108 to compute a differential lag time (e.g., a difference between the cuttings lag time and a gaseous lag time or a difference between the cuttings lag time and a fluid lag time). Absolute cuttings, liquid, and/or gas lag times may also be estimate at 108. It will be appreciated that use of the terms first arrival time and second arrival time is not intended to convey any particular order in time. In other words, the first arrival time is not necessarily earlier than the second arrival time. In fact, in many practical applications the fluid tracer (liquid and/or gas) may arrive at the surface before the solid tracer (such that the second arrival time may be before the first arrival time).

The multiphase tracer may be introduced into the drilling fluid in substantially any suitable manner. For example, the multiphase tracer may be injected in a pulse in which there is negligible time between the beginning and end of the injection. Alternatively, the multiphase tracer may be added to the drilling fluid in a continuous (or near continuous) flow in which the time between the beginning and end of the addition is non-negligible. The tracer concentration in the drilling fluid may be estimated, for example, as a mass feed rate of the tracer divided by the flow rate of the drilling fluid. An arrival time (or arrival time window) and concentration profile of the multiphase tracer at the drill bit may be calculated from the drilling fluid flow rate and the geometry of the drill string using chemical engineering principles known to those of ordinary skill in the art.

With continued reference to FIG. 2, in example embodiments, measuring the first and second arrival times at 104 and 106 may be advantageously performed substantially automatically. For example, as described in more detail below, the rig may be equipped with a degasser (e.g., degasser 54 in FIG. 1) that automatically degasses fluid as it arrives at the surface. The composition of the obtained gases may then be evaluated in real time while drilling for the presence of the liquid tracer or gas tracer (e.g., using known inline gas chromatography techniques). The cuttings and other solid material separated at the shale shaker may be cleaned and evaluated, for example, using digital photographs and a neural network processor to determine whether or not the solid tracer component is present in the cuttings.

As used herein, the term multiphase tracer refers to a tracer that includes at least two material phases or components including at least one solid tracer and at least one fluid tracer. The fluid tracer may be liquid, gas, or supercritical. These two material phases or components are combined into a single tracer component or particle (the multiphase tracer) that may be injected into the drilling fluid. Upon injection, the multiphase tracer (or tracer particles) may travel down the drill string to the drill bit. Upon reaching the bit, the solid tracer and the fluid tracer may be separated from one another (e.g., by the mechanical action of the drill bit) and then transported individually to the surface (e.g., as solid, liquid, or gaseous components).

Upon separation of the multiphase tracer at the drill bit, the solid tracer and the fluid tracer (e.g., the liquid or gaseous tracer), travel upward through the annulus at speeds related to their physical properties (e.g., size, density, solubility, etc.), the fluid physical properties (e.g., viscosity, density), and the geometry of the wellbore annulus. Upon arrival at the surface, the solid and fluid tracer components may be detected using corresponding first and second detection methods to determine corresponding arrival times. The first and second detection methods may be advantageously configured for making continuous or multiple rapid measurements that enable an accurate arrival time determination.

Substantially any suitable detection methods that can be used on a drilling rig may be employed. It will be appreciated that the detection method used may depend upon the physical and chemical properties of the solid and fluid tracers. For example, the solid tracer may be detected using surface-based sensors such as Fourier transformed infrared spectroscopy (FTIR) using, x-ray diffraction (XRD), x-ray fluorescence (XRF), and optical microscopy, video and image recognition, and/or manual detection. Such surface-based measurements may require sample cleaning and drying prior to making the measurements. The solid tracer may also be detected using volume-based sensors such as an ultrasonic sensor, three-dimensional (3D) imaging, nuclear magnetic resonance (NMR), high frequency electromagnetic sensor, or a mud density sensor. Volume sensors do not necessarily require sample preparation and may sometimes be used inline. Moreover, for the solid tracer, the detection method may be configured to detect the presence of the solid tracer particles distributed in drill cuttings of various sizes, shapes, and lithologies. In example embodiments described in more detail below, the solid tracer may be detected by evaluating digital images of drill cuttings using artificial intelligence based digital image processing routines.

The fluid tracer may be detected, for example, using various liquid and/or gas measurement techniques including gas chromatography (GC), high performance liquid chromatography (HPLC), mass spectroscopy (MS), FTIR, XRF, optical microscopy, density determination, video and image recognition, and/or manual detection. In embodiments in which the fluid tracer includes a gaseous component, GC techniques may be advantageous. In embodiments, in which the fluid tracer includes a visually distinguishable (e.g., colored) liquid component, video and image recognition may be advantageous.

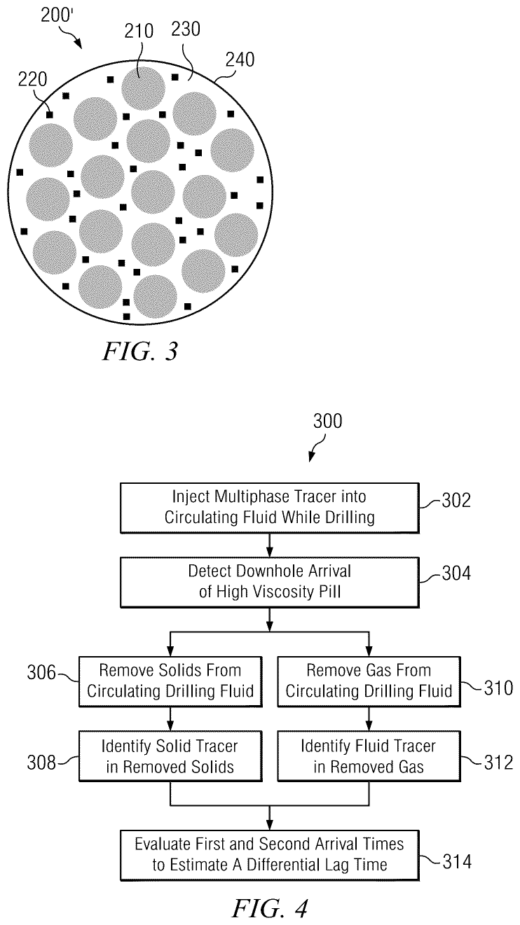

Turning now to FIG. 3, one example of a multiphase tracer 200′ is depicted. In the depicted example embodiment, the multiphase tracer 200′ includes a protective layer 240 enclosing a solid tracer 210, a fluid tracer (e.g., a liquid or gaseous component) 220, and a matrix material 230 in which the solid and fluid components are dispersed or otherwise disposed. It will be appreciated that the disclosed embodiments are not limited to a multiphase tracer as depicted in FIG. 3. For example, the multiphase tracer need not include a protective layer 240 and/or a matrix material 230 so long as it includes a solid tracer and a fluid tracer. Moreover, it will be appreciated that while a spheroidal embodiment is depicted, the multiphase tracer may be any three-dimensional shape, e.g., spheroidal, cylindrical, or cuboidal, as well as largely two-dimensional, such as platelets, or even largely unidimensional, such as fibrous in nature. The disclosed embodiments are not limited in this regard.

The solid tracer 210 may be configured to advantageously have transport properties similar to those of formation cuttings and may further advantageously be easily identified (detected) at the surface amidst a large number of cuttings particles generated while drilling. To promote similar transport properties the solid tracer may have a similar size to drill cuttings, for example, having a largest dimension (such as a diameter) in a range from 0.1 mm to 10 mm (e.g., from 0.2 mm to 2 mm). Moreover, the solid tracer may have a similar density to formation rock, for example, ranging from 1 to 5 g/cm³ (e.g., from 2 to 3 g/cm³). Advantageous solid tracers may further be visually identifiable amidst the drill cuttings to enable the use of automated digital image recognition techniques or visual detection based on colour and/or texture as described in more detail below.

Example solid tracer materials may include carbonate, mica, shale, sandstone, granite, pyrite, metal oxides, metals, ceramic, polymers, and the like. As noted above, the solid tracer may advantageously be easily differentiated from the drill cuttings. Such differentiation may be achieved, for example, via the shape, color, and/or texture of the solid tracer. Moreover, the solid tracer may be coated or dyed to enhance visibility. In example embodiments, the solid tracer may include brightly colored ceramic or polymer beads in the size range described above.

With continued reference to FIG. 3, the fluid tracer 220 may include substantially any suitable liquid and/or gas generating material that can be identified at the surface. In example embodiments the fluid tracer may include a liquid tracer, i.e., a tracer that remains a liquid in the wellbore annulus and that may be utilized to evaluate a drilling fluid lag time. For example, the fluid tracer may include a high viscosity or high temperature boiling point fluid that remains a liquid at the downhole temperature and pressure in the wellbore annulus (as well as at surface temperatures and pressures). Example high viscosity tracers depending on the well conditions may include but are not limited to phenol, naphthalene, xylenes, tetrahydrofuran methylethyl ketone, methyl isobutyl ketone, phenol formaldehyde olygomers, melamine formaldehyde olygomers, bisphenol A, bisphenol F, un-crosslinked epoxy resin monomers, poly amine curing agents, fatty acids, natural oils, and mixtures thereof. Such tracers may be identified using a liquid analysis technique such as liquid chromatography with mass spectroscopy detection, refractive index detection, UV spectroscopy, FTIR, or high temperature gas chromatography.

In other example embodiments, the fluid tracer may include a dye (or an encapsulated dye) configured to color the drilling fluid. A suitable dye may be selected for compatibility with common water-based drilling fluids and formation (connate) water. Advantageous dyes may be stable at the expected wellbore temperatures and not adversely affect any of the physical properties of the drilling fluid. Moreover, the dye may be advantageously selected to have a color that is readily detected by the optical detector (e.g., blue). The liquid tracer may be added in substantially any suitable concentration to provide a dye concentration sufficient to color the drilling fluid (e.g., a dye concentration in a range from about 200 to 2000 mg/L). Advantageous liquid tracers may include Acid Blue #1 dye (EMI-600). Such a dye may be detected using an optical detector deployed in the return flow line.

Alternatively, the fluid tracer may include a low viscosity or low temperature boiling point liquid that evaporates into a gas at ambient pressure and may be identified in the drilling fluid using gas analysis methods such as fast GC or other methods. Such a fluid tracer may be selected such that it tends to remain liquid during transport in the annulus (at higher pressures) and generates detectable quantities of gas at the lower surface pressures. Low viscosity tracers may include, for example, a low molecular weight hydrocarbon, such as saturated, unsaturaturated, aromatic, linear, branched, or cyclic hydrocarbons. The hydrocarbon may be selected so that it can be differentiated from the naturally occurring hydrocarbons in the formation fluid. Such liquid tracers may alternatively and/or additionally include alcohols, ethers, and ketones.

Non comprehensive examples of such liquid tracers depending on the well conditions, include n-butane, n-pentane, isobutane, isopentane, dimethyl propane, n-hexane, n-heptane, n-octane, n-nonane, n-decane, branched hexanes, branched heptanes, branched octanes, branched nonanes, branched decanes, cyclohexane, toluene, benzene, ortho-xylene, meta-xylene, para-xylene, phenol, cresol, ethanol, propanol, isopropanol, butanol, isobutanol, dioxane, tetrahydrofuran, acetone, methyl-ethyl ketone, dichloromethane, chloroform, ethyleneglycol, propylene glycol, ethyleneglycol monomethyl ether, ehtylenglycol monoethyl ether, ethylenglycol monobutyl ether, diethyleneglycol monomethyl ether, diethyleneglycol monoethyl ether, diethylene glycol monobutylether, and the like, and mixtures thereof.

In other example embodiments, the fluid tracer may include a gaseous tracer or a tracer that decomposes into a gas in the wellbore annulus. Such gaseous tracers may be either solid or liquid so long as they decompose into a gas in the drilling fluid. Gaseous tracers may include, for example, calcium carbide, urea, methyl urea, metal sulfites or bisulfites, metal carbonates, dimethyl carbonate, and/or water reactive metals such as aluminum, magnesium, calcium, strontium, sodium, potassium, and alloys thereof that upon exposure to water (in a water-based drilling fluid) may release hydrogen gas (due to oxidation of the metal). Such gaseous tracers may be identified at the surface using gas analysis methods such as fast GC or other methods.

With still further reference to FIG. 3 the matrix material 230, when used, is intended to disperse and support the solid tracer 210 and fluid tracer 220 in the multiphase tracer. The matrix material may solid or liquid and may be selected to readily shear at the bit thereby allowing the solid tracer and fluid tracer to be dispersed into the drilling fluid. Example matrix materials may be selected from clay powder, silica powder, talc powder, carbonate powder, polymer or plastic powder, liquid or semi liquid epoxy resins, phenolic resins, melamine resins, and their curing agents, oils, waxes, and mixtures thereof.

The protective layer 240, when used, is intended to further protect and support the solid tracer 210 and fluid tracer 220 in the multiphase tracer, particularly during transit to the bit. The protective layer may be readily sheared to torn at the bit to release the solid and fluid tracers. Example protective layers may include a metal foil such as aluminum foil, a glass or fiberglass film or layer, a polymer or plastic layer, crosslinked epoxy resin, phenolic resin, and/or melamine resin, polycarbonate, polymethylmethacrylate, and the like.

It will further be appreciated that the multiphase tracer may be fabricated using substantially any suitable methods. Example, methods of fabrication may include extrusion, injection molding, casting, 3D printing, milling followed by cutting, dry coating, and spray coating, depending on the nature of the components. For example, a multiphase tracer including a fluid tracer and a solid tracer may be fabricated by admixing the tracer components in a vessel, e.g., according to a suitable mass balance, followed by injection into a casting mold, curing, and drying. Alternatively, spray coating a latex containing the fluid tracer, and the chemicals producing the protective layer 240 of a solid particulate, followed by curing and drying may be used as a method to manufacture a different multiphase tracer. Moreover, a coextrusion of the solid tracer and fluid tracer in the presence of an agglomerating matrix material, followed by extrudate cutting may be used to produce the multiphase tracer.

With continued reference to FIGS. 2 and 3, a non-limiting example multiphase tracer may include a ceramic, polymeric, silica, and/or bauxite particle having a diameter of about 1 mm to 3 mm (US mesh size 7 to 18). The particle may be coated with a curable phenolic resin coating, for example, containing an excess of free phenol (e.g., applied as a sprayable latex or an epoxy resin containing an excess of bisphenol A). The coated particle may then be cured. Such a multiphase tracer may include a solid tracer (the particle) and a combined fluid tracer/protective layer in which the cured phenolic resin acts as a protective layer and the free phenol acts a fluid tracer. Moreover, depending on the nature of the coating material selected, the particle may include a distinct colour such as yellow, green or red, which may be distinguishable from formation cuttings.

FIG. 4 depicts a flow chart of another example method 300 for estimating a lag time or differential lag time while drilling. The method includes introducing a multiphase tracer into the circulating drilling fluid at 302. The multiphase tracer may be injected with (e.g., immediately before or in) a high viscosity pill (high viscosity drilling fluid). As described above, the multiphase tracer may include substantially any suitable tracer including a solid tracer and a fluid tracer. Method 300 further includes detecting the downhole arrival of the high viscosity pill at 304. For example, the downhole arrival of the high viscosity pill may be detected by a change or pulse in standpipe pressure at the surface as the pill passes through the drill bit jets or by a downhole pressure change or pulse (e.g., measured using a downhole pressure sensor). The arrival of the high viscosity pill may alternative be measured using downhole logging measurements that are sensitive to changes in drilling fluid properties, for example, via a change in the mud resistivity as measured using a resistivity LWD tool or by a change in a fluid relaxation time as measured using NMR.

Upon passing through the drill bit jets, the multiphase tracer is configured to release the solid tracer and the fluid tracer as described above. Solids are removed from the drilling fluid arriving at the surface at 306 (e.g., via a shale shaker). The solids may include drill cuttings and the solid tracer (upon its arrival at the surface). A first arrival time of the solid tracer is determined at 308 by evaluating digital images of the removed solids (e.g., including drill cuttings and the solid tracer). As described in more detail below, the solid tracer may be identified in the digital images using automated artificial intelligence routines (e.g., via a trained neural network). Gas is removed (e.g., extracted) from the drilling fluid at 310 (e.g., via a degasser in the return flow line). The gas may include formation gases and one or more fluid tracer gases. A second arrival time of the fluid tracer is determined at 312 by evaluating a composition of the obtained gas. As described in more detail below, the gas may be evaluated using a GC or other inline technique. The first and second arrival times are then evaluated at 314 to compute a differential lag time (e.g., a difference between the cuttings lag time and the gas or liquid lag time). Absolute cuttings, liquid, and/or gas lag times may also be computed at 314 by further evaluating the downhole arrival of the high viscosity pill.

With continued reference to FIG. 4, digital images of the solids may be acquired by removing the solids from the drilling fluid, preparing the solids, and taking a digital photograph thereof. The solids may be obtained, for example, from the shale shakers (or other solids control equipment) or by screening drilling fluid from a return mud pit or a return flow line. Those of ordinary skill will readily appreciate that a return mud pit is a mud pit to which drilling fluid returns after passing through the shale shakers and/or other solids control equipment. Samples obtained from the shale shakers or other solids control equipment may include drill cuttings and may further include solid tracer particles when the solid tracer has arrived back at the surface.

The solids may be prepared for image analysis, for example, by washing and then drying in an oven. In certain embodiments, such as when samples are obtained from the shake shakers or solids control equipment, the sample preparation may also include sieving or meshing solids to remove large and/or small particles (e.g., to remove a portion of the cuttings particles). The solids may be further placed in a tray having a high contrast (vivid) background color to enhance subsequent particle identification and segmentation in the acquired images, for example, pure magenta (e.g., with RGB values of 255, 0, 255), pure blue (e.g., with RGB values of 0, 0, 255), pure green (e.g., with RGB values of 0, 255, 0), and so forth. In general, such colors do not exist in nature and, accordingly, may help the segmentation algorithm to avoid detecting the background of the tray as part of the particle. The tray of prepared particles may be placed in front of a digital camera and at least one digital image may be taken, for example, a white light image, or a first white light image and a second infrared or ultraviolet image, or even a first white light image, a second infrared image, and a third ultraviolet image. The disclosed embodiments are not limited in these regards; however, it will be appreciated that the acquisition of multiple images may be advantageous in that certain texture features may be more readily discerned in infrared or ultraviolet light than in white light.

In certain embodiments, the image acquisition process may advantageously make use of standardized and/or calibrated lighting, color enhancement, magnification, and/or focus/resolution settings. For example, in certain embodiments, color/illumination calibration is obtained by using colorimetry algorithms against previously analyzed photos and a current photo of interest, while resolution calibration may be based on lens focal length, focal distance, and sensor size/resolution for the current photo of interest as compared to that of previously analyzed photos. Images may be taken when the cuttings are wet or dry, with the humidity generally being controlled for dry cuttings images.

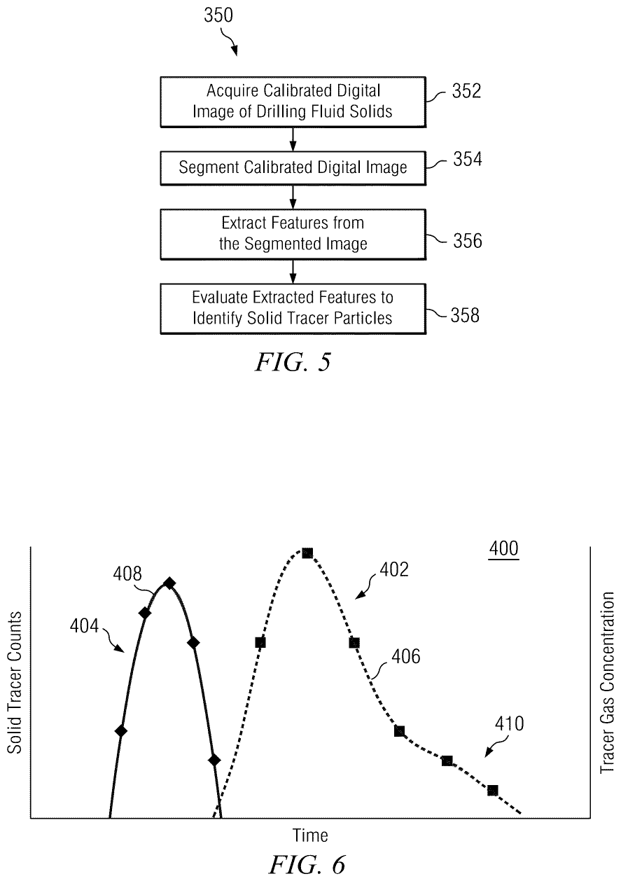

Turning now to FIG. 5, one example method 350 for determining the first arrival time by evaluating digital images is depicted. A calibrated digital image may be acquired at 352, for example, as described above. The calibrated digital image may be processed with a segmenting algorithm to obtain segmented images at 354. The segmenting algorithm may be configured, for example, to identify individual particles (e.g., cuttings particles and solid tracer particles) in the calibrated images. A segmenting algorithm may employ a Mask Region-Based Convolutional Neural Network (Mask R-CNN) such as disclosed in U.S. patent application Ser. No. 17/647,407. The Mask R-CNN may be configured to identify various objects (such as individual cuttings and solid additive particles) in the digital images and thereby generate the segmented image at 354. The Mask R-CNN may produce, for example, bounding boxes and mask images. The bounding boxes may be defined as a set of x-y coordinates in an image that indicates an image region that contains an object of interest. The bounding box may include a confidence score that ranges from 0 to 1 (e.g., with greater values indicating higher confidence regarding) for each object of interest. The mask image may indicate (e.g., highlight or otherwise bound) regions of interest that have a confidence score that exceeds a threshold.

The segmented image may be further processed at 356 to extract texture and/or color features from one or more of the identified particles in the segmented image. For example, the image may be evaluated particle by particle to extract the color, texture and/or size and shape related features thereof. Various color related features may include, for example, average (such as mean, median, or mode) red, green, and blue intensities or distributions of or standard deviations of red, green, and blue intensities and/or an average luminance of each particle as well as a histogram, a variance, a skewness, and/or a kurtosis of the red, green, and blue intensities. Texture related features quantify spatial relationships and/or directional changes in pixel color and/or brightness in each particle. Example extracted texture related features may include, for example, edge detection, pixel to pixel contrast, correlation, and/or entropy. Example size and shape related features may include, for example, a particle diameter, an area, a perimeter, a maximum axis, a minimum axis, a particle aspect ratio, and internal angle measurements. Moreover, spatial relationships of the pixels grouped in each particle may be evaluated to extract particle circularity, solidity, elongation, roundness, and/or convex hull area.

The extracted features may be processed at 358 to identify (or distinguish) solid tracer particles from among the drill cuttings. The first arrival time (the solid tracer arrival) may be determined from the first identification or when a proportion (or fraction) of solid tracer particles exceeds a threshold. The solid tracer particles may be identified, for example, according to a location of the particle in a multi-dimensional space of extracted color, texture, size, and/or shape features. For example, as described above, a set of color and texture features may be computed (e.g., for selected ones segmented particles). The set of computed color and texture features may include a large number of features, for example, including at least 16 features (e.g., at least 32, 48, 64, 80, 96, 112, or 128 color and texture features). The particles may then be classified (e.g., as cuttings or solid tracer particles) according to values of those features, for example, that cause like particles to cluster in the aforementioned multi-dimensional feature space.

With reference again to FIG. 4, gas may be removed (e.g., extracted) from the drilling fluid at 310 using a degasser in the return flow line. The gas generally includes formation gases and may further include fluid tracer gases (e.g., after the arrival of the fluid tracer at the surface). The extracted gas may be piped to an onsite laboratory 60 (FIG. 1), for example, to automatically transport the sampled gases to a testing apparatus. The testing apparatus may evaluate the gas composition at some regular interval while drilling. The arrival of the fluid tracer gas may be observed as a change in the composition of the gas sample, for example, a marked change in the concentration of one or more compounds in the gas sample or as the arrival of a new gas compound.

With still further reference to FIGS. 2, 4, and 5, it will be understood that the disclosed embodiments may further enable a lag time distribution (or profile) to be estimated. In such embodiments, identification of an asymmetric profile may advantageously enable potential cuttings transport issues to be identified and remedied before poor transport causes more serious drilling issues. In one example embodiment, the lag time profile may be evaluated by evaluating the number of detected solid tracer particles with time during a drilling operation. The detected tracer gas concentration may also be evaluated with respect to time. FIG. 6 depicts one example plot 400 of solid tracer counts (vertical left axis) and tracer gas concentration (vertical right axis) versus time (horizontal axis). In the depicted plot the solid tracer counts are depicted as squares at 402 and the tracer gas concentrations are depicted as diamonds at 404. Corresponding best fits are shown at 406 and 408. In the depicted example, the solid tracer profile is asymmetric at long times as indicated at 410 and may indicate potential hole cleaning issues.

It will be understood that the present disclosure includes numerous embodiments. These embodiments include, but are not limited to, the following embodiments.

In a first embodiment, a method for estimating a differential lag time while drilling includes introducing a multiphase tracer into drilling fluid circulating in a wellbore while drilling, the multiphase tracer including a solid tracer and a fluid tracer, the fluid tracer including at least one of a liquid tracer or a gaseous tracer; measuring a first arrival time of the solid tracer at a surface location; measuring a second arrival time of the fluid tracer at the surface location; and evaluating the first and second arrival times to estimate the differential lag time, wherein the differential lag time includes at least one of a difference between a cuttings lag time and a gaseous lag time or a difference between the cuttings lag time and a liquid lag time.

A second embodiment may include the first embodiment, wherein the solid tracer and the fluid tracer are disposed in a matrix material that is enclosed in a protective layer.

A third embodiment may include any one of the first through second embodiments, wherein the multiphase tracer comprises a solid ceramic, polymeric, silica, or bauxite particle embedded in a curable phenolic resin coating including excess free phenol.

A fourth embodiment may include any one of the first through third embodiments, wherein the solid tracer comprises solid particles having a largest dimension in a range from 0.1 mm to 10 mm and a density in a range from 1 to 5 g/cm³.

A fifth embodiment may include any one of the first through fourth embodiments, wherein the fluid tracer is a liquid tracer that remains liquid during transport to the surface location and then generates detectable quantities of a gas at the surface location.

A sixth embodiment may include any one of the first through fifth embodiments, wherein the fluid tracer comprises a solid material or a liquid material that evaporates into a gas tracer after passing through drill bit jets into an annulus of the wellbore.

A seventh embodiment may include any one of the first through sixth embodiments, wherein the first arrival time is measured by automatically identifying the solid tracer in digital images using a trained neural network; and the second arrival time is measured by automatically identifying the fluid tracer as a composition change of gas obtained from the drilling fluid.

An eighth embodiment may include any one of the first through seventh embodiments, wherein the measuring the first arrival time comprises removing solids from the circulating drilling fluid; acquiring digital images of the removed solids; and evaluating the digital images with a trained neural network to identify the solid tracer.

A ninth embodiment may include any one of the first through eighth embodiments, wherein the measuring the second arrival time comprises extracting gas from the circulating drilling fluid using a degasser; evaluating a composition of the extracted gas; and identifying the fluid tracer by a change in the composition of the gas.

A tenth embodiment may include any one of the first through ninth embodiments, wherein the multiphase tracer is introduced into the circulating drilling fluid in a high viscosity pill; and the method further comprises measuring a downhole arrival time of the high viscosity pill.

In an eleventh embodiment, a system for estimating a differential lag time while drilling includes a plurality of multiphase tracer particles configured for introduction into drilling fluid circulating in a wellbore while drilling, each of the plurality of multiphase tracer particles including a solid tracer and a fluid tracer, the fluid tracer including at least one of a liquid tracer or a gaseous tracer; a first detector configured to measure a first arrival time of the solid tracer at a surface location; a second detector configured to measure a second arrival time of the fluid tracer at the surface location; and a processor configured to evaluate the first and second arrival times to estimate the differential lag time, wherein the differential lag time includes at least one of a difference between a cuttings lag time and a gaseous lag time or a difference between the cuttings lag time and a liquid lag time.

A twelfth embodiment may include the eleventh embodiment, wherein each of the plurality of multiphase tracer particles comprises the solid tracer and the fluid tracer disposed in a matrix material that is enclosed in a protective layer.

A thirteenth embodiment may include any one of the eleventh through twelfth embodiments, wherein each of the plurality of multiphase tracer particles comprises a solid ceramic, silica, or bauxite particle embedded in a curable phenolic resin coating including excess free phenol.

A fourteenth embodiment may include any one of the eleventh through thirteenth embodiments, wherein the first detector comprises a digital camera configured to take digital images of solids removed from the circulating drilling fluid; and a processor configured to automatically identify the solid tracer in the digital images using a trained neural network.

A fifteenth embodiment may include any one of the eleventh through fourteenth embodiments, wherein the second detector comprises a detector configured to evaluate a composition of gas extracted from the circulating drilling fluid; and a processor configured to automatically identify the fluid tracer as a composition change of the extracted gas.

In a sixteenth embodiment, a method for estimating a differential lag time while drilling includes introducing a multiphase tracer into drilling fluid circulating in a wellbore while drilling, the multiphase tracer introduced before or in a high viscosity pill, the multiphase tracer including a solid tracer and a fluid tracer, the fluid tracer including at least one of a liquid tracer or a gaseous tracer; measuring a downhole arrival time of the high viscosity pill; removing solids from the circulating drilling fluid at a surface location; evaluating digital images of the removed solids to identify a first arrival time of the solid tracer at the surface location; extracting gas from the circulating drilling fluid using a degasser at the surface location; evaluating a composition of the extracted gas to identify a second arrival time of the fluid tracer at the surface location; and evaluating the first arrival time and the second arrival time to estimate the differential lag time, wherein the differential lag time includes at least one of a difference between a cuttings lag time and a gaseous lag time or a difference between the cuttings lag time and a liquid lag time.

A seventeenth embodiment may include the sixteenth embodiment, wherein the multiphase tracer comprises a solid ceramic, silica, or bauxite particle embedded in a curable phenolic resin coating including excess free phenol.

An eighteenth embodiment may include any one of the sixteenth through seventeenth embodiments, wherein the fluid tracer is a liquid tracer that remains liquid during transport to the surface location and then generates detectable quantities of a gas at the surface location; or wherein the fluid tracer comprises a solid or liquid material that evaporates into a gas tracer after passing through drill bit jets into an annulus of the wellbore.

A nineteenth embodiment may include any one of the sixteenth through eighteenth embodiments, wherein the evaluating the digital images comprises automatically identifying the solid tracer in digital images using a trained neural network; and the evaluating the composition comprises automatically identifying the fluid tracer as a composition change of gas obtained from the drilling fluid.

A twentieth embodiment may include any one of the sixteenth through nineteenth embodiments, wherein the evaluating the first arrival time and the second arrival time further comprises evaluating the downhole arrival time of the high viscosity pill to estimate a cuttings lag time and at least one of a liquid lag time and a gas lag time.

Although automated methods for estimating differential lag times while drilling have been described in detail, it should be understood that various changes, substitutions and alternations can be made herein without departing from the spirit and scope of the disclosure as defined by the appended claims.

What is claimed is:

1. A method for estimating a differential lag time while drilling, the method comprising:
introducing a multiphase tracer into drilling fluid circulating in a wellbore while drilling, the multiphase tracer including a solid tracer and a fluid tracer, the fluid tracer including at least one of a liquid tracer or a gaseous tracer;
measuring a first arrival time of the solid tracer at a surface location;
measuring a second arrival time of the fluid tracer at the surface location; and
evaluating the first and second arrival times to estimate the differential lag time, wherein the differential lag time includes at least one of a difference between a cuttings lag time and a gaseous lag time or a difference between the cuttings lag time and a liquid lag time.

2. The method of claim 1, wherein the solid tracer and the fluid tracer are disposed in a matrix material that is enclosed in a protective layer.

3. The method of claim 1, wherein the multiphase tracer comprises a solid ceramic, polymeric, silica, or bauxite particle embedded in a curable phenolic resin coating including excess free phenol.

4. The method of claim 1, wherein the solid tracer comprises solid particles having a largest dimension in a range from 0.1 mm to 10 mm and a density in a range from 1 to 5 g/cm³.

5. The method of claim 1, wherein the fluid tracer is a liquid tracer that remains liquid during transport to the surface location and then generates detectable quantities of a gas at the surface location.

6. The method of claim 1, wherein the fluid tracer comprises a solid material or a liquid material that evaporates into a gas tracer after passing through drill bit jets into an annulus of the wellbore.

7. The method of claim 1, wherein:
the first arrival time is measured by automatically identifying the solid tracer in digital images using a trained neural network; and
the second arrival time is measured by automatically identifying the fluid tracer as a composition change of gas obtained from the drilling fluid.

8. The method of claim 1, wherein the measuring the first arrival time comprises:
removing solids from the circulating drilling fluid;
acquiring digital images of the removed solids; and
evaluating the digital images with a trained neural network to identify the solid tracer.

9. The method of claim 1, wherein the measuring the second arrival time comprises:
extracting gas from the circulating drilling fluid using a degasser;
evaluating a composition of the extracted gas; and
identifying the fluid tracer by a change in the composition of the gas.

10. The method of claim 1, wherein:
the multiphase tracer is introduced into the circulating drilling fluid in a high viscosity pill; and
the method further comprises measuring a downhole arrival time of the high viscosity pill.

11. A system for estimating a differential lag time while drilling, the system comprising:
a plurality of multiphase tracer particles configured for introducing into drilling fluid circulating in a wellbore while drilling, each of the plurality of multiphase tracer particles including a solid tracer and a fluid tracer, the fluid tracer including at least one of a liquid tracer or a gaseous tracer;
a first detector configured to measure a first arrival time of the solid tracer at a surface location;
a second detector configured to measure a second arrival time of the fluid tracer at the surface location; and
a processor configured to evaluate the first and second arrival times to estimate the differential lag time, wherein the differential lag time includes at least one of a difference between a cuttings lag time and a gaseous lag time or a difference between the cuttings lag time and a liquid lag time.

12. The system of claim 11, wherein each of the plurality of multiphase tracer particles comprises the solid tracer and the fluid tracer disposed in a matrix material that is enclosed in a protective layer.

13. The system of claim 11, wherein each of the plurality of multiphase tracer particles comprises a solid ceramic, silica, or bauxite particle embedded in a curable phenolic resin coating including excess free phenol.

14. The system of claim 11, wherein the first detector comprises:
a digital camera configured to take digital images of solids removed from the circulating drilling fluid; and
a processor configured to automatically identify the solid tracer in the digital images using a trained neural network.

15. The system of claim 11, wherein the second detector comprises:
a detector configured to evaluate a composition of gas extracted from the circulating drilling fluid; and
a processor configured to automatically identify the fluid tracer as a composition change of the extracted gas.

16. A method for estimating a differential lag time while drilling, the method comprising:
introducing a multiphase tracer into drilling fluid circulating in a wellbore while drilling, the multiphase tracer introduced before or in a high viscosity pill, the multiphase tracer including a solid tracer and a fluid tracer, the fluid tracer including at least one of a liquid tracer or a gaseous tracer;
measuring a downhole arrival time of the high viscosity pill;
removing solids from the circulating drilling fluid at a surface location;
evaluating digital images of the removed solids to identify a first arrival time of the solid tracer at the surface location;
extracting gas from the circulating drilling fluid using a degasser at the surface location;
evaluating a composition of the extracted gas to identify a second arrival time of the fluid tracer at the surface location; and
evaluating the first arrival time and the second arrival time to estimate the differential lag time, wherein the differential lag time includes at least one of a difference between a cuttings lag time and a gaseous lag time or a difference between the cuttings lag time and a liquid lag time.

17. The method of claim 16, wherein the multiphase tracer comprises a solid ceramic, silica, or bauxite particle embedded in a curable phenolic resin coating including excess free phenol.

18. The method of claim 16, wherein:
the fluid tracer is a liquid tracer that remains liquid during transport to the surface location and then generates detectable quantities of a gas at the surface location; or
wherein the fluid tracer comprises a solid or liquid material that evaporates into a gas tracer after passing through drill bit jets into an annulus of the wellbore.

19. The method of claim 16, wherein:
the evaluating the digital images comprises automatically identifying the solid tracer in digital images using a trained neural network; and
the evaluating the composition comprises automatically identifying the fluid tracer as a composition change of gas obtained from the drilling fluid.

20. The method of claim 16, wherein the evaluating the first arrival time and the second arrival time further comprises evaluating the downhole arrival time of the high viscosity pill to estimate a cuttings lag time and at least one of a liquid lag time and a gas lag time.
